(19) United States
(12) Patent Application Publication
(10) Pub. No.: US 2023/0145641 A1
(43) Pub. Date: May 11, 2023

(54) AN ILLUMINATION DEVICE FOR TEAR FILM LIPID LAYER ON OCULAR SURFACE ATTACHED TO AN OPHTHALMIC SLIT LAMP MICROSCOPE

(71) Applicant: CHONGQING KANGHUA RUIMING SCIENCE TECHNOLOGY CO., LTD., Chongqing (CN)

(72) Inventor: Yi Wang, ChongQing (CN)

(21) Appl. No.: 18/052,209

(22) Filed: Nov. 3, 2022

(30) Foreign Application Priority Data
Nov. 11, 2021 (CN) 202111331343.2

(51) Int. Cl. A61B 3/00; A61B 3/135; A61B 3/10

(52) U.S. Cl. CPC A61B 3/0008; A61B 3/0075; A61B 3/101; A61B 3/135

(57) ABSTRACT

The utility model relates to an illumination device (100) for tear film lipid layer on ocular surface attached to an Ophthalmic Slit lamp Microscope, which comprises a ophthalmic slit lamp microscope. An illumination device (100) for tear film lipid layer is installed on the slit lamp microscope. The illumination device (100) is located at the lower part of the observer’s eye to illuminate the surface of the lower half of the eye surface angular membrane of the observer’s eye for observing the tear film lipid layer. The light emitted by the light source of the eye surface tear film lipid layer illumination device is inclined relative to the eye axis direction (10), and its inclination angle is ≥ 20°, which reduces the noise interference of transparent parts such as iris behind the cornea, reduces the noise, and improves the observation and imaging clarity of the eye surface lipid layer.

### TECHNICAL FIELD

The patent relates to the field of special medical equipment, in particular to a lighting device for tear film lipid layer on ocular surface attached to an Ophthalmic Slit lamp Microscope.

### BACKGROUND TECHNOLOGY

The eye is the most important organ in human senses. About 80% of the knowledge in the brain is obtained through the eye. Eyes should be used to read, read pictures, figure and enjoy the beautiful scenery.

Ophthalmic Slit lamp microscope (referred to as slit lamp for short) is not only an important instrument for ophthalmic examination, but also it is the first widely used basic instrument for routine ophthalmic examination. Ophthalmic Slit lamp is mandatory in ophthalmic diagnosis and treatment, ophthalmic health care, optometry and glasses and other industries.

Worldwide, from the smallest outpatient department of facial features to large ophthalmic hospitals to top ophthalmic disease research institutions, ophthalmic slit lamp microscope is the most professional equipment to examine eyelids, conjunctiva, sclera, cornea, anterior chamber, iris, pupil, lens and anterior ⅓ vitreous to determine the location, nature, size and depth of lesions.

Ophthalmic slit lamp microscope is widely used by medical institutions, ophthalmic health care institutions and chain of glasses stores. The expansion and improvement of the function of the instrument plays an important role in human eye health.

The structure of ophthalmic slit lamp is mainly composed of four parts: slit lighting mechanism, microscope optical body, mobile platform mechanism and cheek support mechanism. Among them, the most important two parts are slit lighting mechanism and microscope optical body.

To expand the function of slit lamp and add functional accessories, For example, a dry eye projection (illumination) device for dry eye examination (an ophthalmic disease) is added. The dry eye corneal projection (illumination) device (hereinafter referred to as the dry eye device) is attached to the slit lamp and placed between the eye and the slit lamp during operation. The optical axis of the dry eye device coincides with the imaging optical axis of the slit lamp, and the operator uses the device to illuminate the eye surface (visible light and infrared light illumination) or projection illumination pattern on the ocular surface (Placido ring, etc.). The device is an optical projection device with horn shaped opening, and its irradiation angle to the ocular surface is ≤ 15°.

Tear film is a protective film on the surface of the human eye that separates the cornea from the air. The tear film consists of three layers, the inner layer is mucin, themiddle layer is tear, and the outer layer is lipid layer. The distribution of lipid layer on the tear film surface is generally uneven, irregular and dynamic.

By blinking, people smear tears and lipid layer on the ocular surface secreting mucin to form a tear film. The tear film ruptures in about ten seconds under normal conditions. After a large number of ruptures, the tear film needs to be reconstructed by blinking. When the tear film changes, the rupture time becomes faster. The human eye surface lacks tear film protection, and long-term direct contact with the air will produce discomfort, which is called dry eye.

Dry eye is the most common and ocular disease with high incidence rate. The incidence rate of 20~36%/ in China is increasing. The development trend in recent years is increasing obviously with the increase of eye frequency. The study of this disease is of great importance to human health.

The causes of dry eye are divided into: mucin problem, tear problem and lipid layer problem. The problem of lipid layer causes dry eye, which is caused by insufficient or abnormal lipid layer, which can not cover the tear surface well, make the tear film evaporate too fast and rupture too early, and can not lock the water, resulting in dry eye.

The existing slit lamp is used for dry eye surface lipid layer inspection, and the placido ring light or ordinary visible light irradiated by an additional dry eye device is used for eye surface lipid layer illumination. The disadvantages of using the dry eye device to check the eye surface lipid layer are: a. the dry eye device is an optical projection device with a horn opening and its effective irradiation part is around the pupil of the eye, and the irradiation area is small, which can not completely reflect the actual situation of the whole eye surface lipid layer; b. the light of Placido ring irradiated by the dry eye device with Placido ring interferes with the observation of eye surface lipid layer and affects the observation effect; c. the light irradiated by the dry eye device is similar to the small angle illumination perpendicular to the ocular surface (the angle irradiated to the ocular surface is ≤ 15 °), and the optical signal will be interfered by the reflected light and noise from the non observed tissues behind the cornea such as iris and pupil. Because the cornea and its surface attachments (such as tear film and lipid layer on tear film) are transparent, these interference noises are much stronger than the optical signal on the corneal surface (i.e. extremely low signal-to-noise ratio), it is impossible to observe the tear film lipid layer well at all, so it is necessary to suppress interference and improve signal strength to achieve the purpose of effectively observing the tear film lipid layer; and d. the dry eye device is only about 20 mm away from the eyes at the working position, and the distance is too small for the operator to turn over the lower eyelid by hand.

### SUMMARY OF THE INVENTION

Aiming at the shortcomings of the existing technology, the present invetion proposes an illumination device for tear film lipid layer on ocular surface attached to an Ophthalmic Slit lamp Microscope.

Aiming at the shortcomings of the prior art, the presention invention discloses an eye surface tear film lipid layer illumination device attached to the ophthalmic slit lamp microscope. The specific technical scheme is as follows:

An illumination device for tear film lipid layer on ocular surface attached to an Ophthalmic Slit lamp Microscope, which comprises a Ophthalmic slit lamp microscope, which is characterized in that a left and right rotation mechanism is hinged on the optical body of the ophthalmic slit lamp microscope.

In an embodiment, the present invention provides an illumination device for tear film lipid layer on ocular surface is installed on the left and right rotation mechanism, which is located at the lower part of the subject’s eye to illuminate the surface of the lower half of the subject’s eye surface cornea for observing the condition of the tear film lipid layer;
said illumination device for tear film lipid layer on ocular surface comprises a connecting part and an illumination body, one end of the connecting part is connected with the left and right rotation mechanism, the other end of the connecting part is connected with the illumination body, and the illumination body light source is a surface light source; and
a front and rear adjusting mechanism is installed on the left and right rotation mechanism, and a dry eye device is installed on the sliding part of the front and rear adjusting mechanism.

In order to better realize the utility model, it can be further as follows: the light emitted by the light source of the eye surface tear film lipid layer lighting device is inclined relative to the eye axis direction, and the inclination angle is ≥ 20°.

Further, the illumination body comprises a translucent flood board, a lipid layer lighting device/ LED matrix lamp group and a lipid layer lighting device housing, the lipid layer lighting device/ LED matrix lamp group is installed on the inner surface of the lipid layer lighting device housing, and a translucent flood board is installed on the lipid layer lighting device housing.

Further, the optical light emitted by the LED matrix lamp group of the lipid layer lighting device is diffused through the translucent flood board, transformed into a soft and uniform area light source, and mapped to the lower half of the cornea on the ocular surface of the eye.

The beneficial effects of the present invention are:

First, the lipid layer lighting device irradiates the corneal surface at a large tilt angle (tilt angle ≥ 20 °), which reduces the noise interference of transparent parts such as iris behind the cornea, reduces the noise, and improves the observation and imaging clarity of ocular surface lipid layer.

Second, the working position of the lipid layer lighting device is farther away from the eyes than when the dry eye device is used for lighting, which is convenient for the operator to observe and turn over the eyelids of the inspected person.

Third, through the analysis of the high-quality pictures obtained after the eye surface lipid layer is irradiated by the lipid layer lighting device, the thickness of different parts of the eye surface lipid layer can be measured accurately, and then the average lipid layer thickness (LLT) can be calculated. Through these data, it is convenient to obtain a more accurate diagnosis conclusion.

Fourth, the structure installed on the dry eye device is compact. When in use, the back movement of the dry eye device will not block the light of the lipid layer lighting device. It can also rotate to the non working position with the dry eye device, which will not affect the realization of other functions of the ophthalmic slit lamp.

### DESCRIPTION OF DRAWINGS

FIG. 1 is a side view of the lighting device of the present patent;

FIG. 2 is a front view of the lighting device of the present patent;

FIG. 3 is a general schematic diagram of the present patent;

The reference numerals in the figure illustrate illumination device (100) for tear film lipid layer on ocular surface, eye 1, dry eye device 2, translucent flood board 3, lipid layer lighting device/ LED matrix lamp group 4, lipid layer lighting device housing 5, front and rear adjusting mechanism 6 of the dry eye device (i.e. the front and back adjusting mechanism) of the dry eye device 2, the left and right rotation mechanism 7 (i.e. left and right rotation mechanism), the slit lamp microscope optical body 8, the optical direction 9 of the lipid layer illumination device, and the eye axis direction 10 (the eyes of the observer front squarely, the eye axis direction).

### DETAILED DESCRIPTION OF THE INVENTION

The preferred embodiment of the patent is described in detail below in combination with the accompanying drawings, so that the advantages and features of the patent can be more easily understood by those skilled in the art, so as to make a clearer and clear definition of the protection scope of the patent.

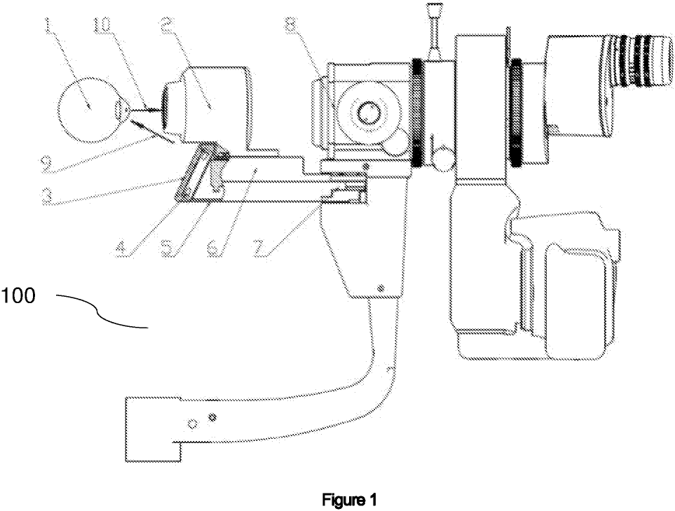

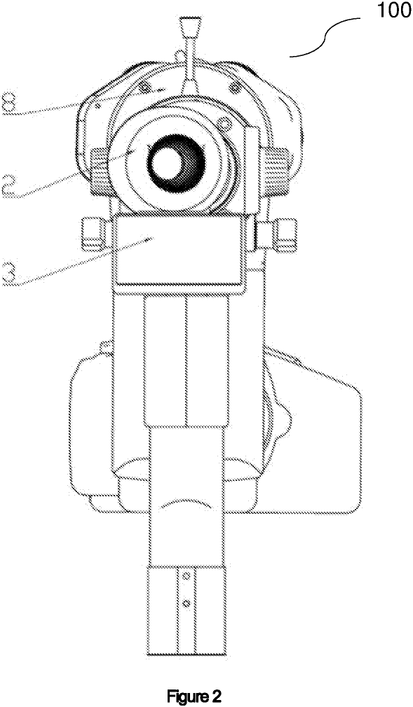

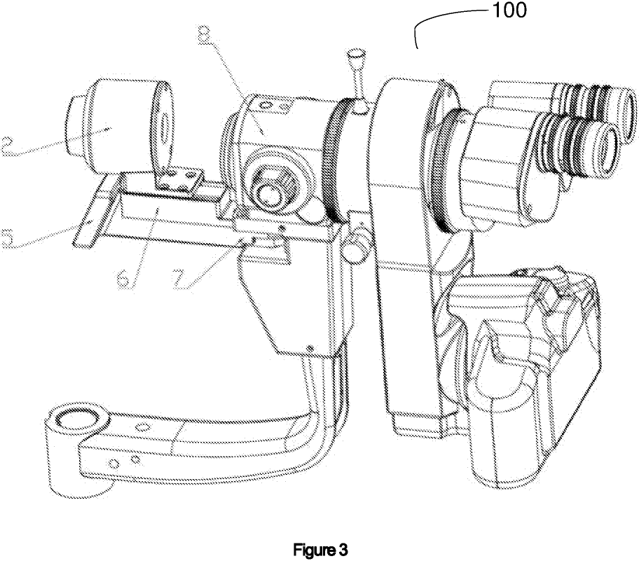

As shown in FIGS. 1 to 3, an illumination device (100) for tear film lipid layer on ocular surface is installed on optical body 8 of the ophthalmic slit lamp microscope, which is located at the lower part of the observer’s eye 1; the optical direction 9 emitted by the light source of the eye surface tear film lipid layer illumination device is inclined relative to the eye axis direction 10 (the direction of the eye axis facing the front of the observed eye), and the inclination angle is 25° ~ 80°.

Four groups of LED matrix lamp groups 4 are arranged along the inner wall of the lipid layer lighting device housing 5, the translucent flood board 3 is installed on the lipid layer lighting device housing 5, and the other end of the lipid layer lighting device housing 5 is connected to the dry eye device 2 attached to the slit lamp microscope optical body 8. The function of the translucent flood board 3 is to diffuse the light of the LED matrix lamp group 4 into a soft and uniform surface light source, so that the irradiation surface mapped to the lower half of the cornea of the eye surface is uniform and clear.

In this embodiment, the lipid layer lighting device 4 is fixed at the end of the left and right rotation mechanism 7 of the dry eye device 2 attached to the slit lamp microscope optical body 8.

The left and right rotation mechanism 7 is articulated on the optical body 8 of the slit lamp microscope, and a front and rear adjusting mechanism 6 is arranged on the left and right rotation mechanism 7, and a dry eye device 2 is arranged on the sliding part of the front and rear adjusting mechanism 6.

The additional dry eye device 2 of the slit lamp microscope can be moved back and forth. When the lipid layer lighting device 4 is used, the additional dry eye device 2 of the slit lamp microscope retreats towards the slit lamp microscope optical body 8, so that the dry eye device 2 does not block the optical direction 9 emitted by the lipid layer lighting device 4. When the lipid layer lighting device 4 is not in use, the dry eye device 2 attached to the slit lamp microscope can rotate more than 90° to the left (or right) to exit the optical body imaging optical path of the slit lamp microscope, which will not affect the realization of other functions of the slit lamp microscope.

The illumination light emitted by the LED matrix lamp group 4 irradiates on the translucent flood board 3 to form an area light source. The area light source irradiates the corneal surface in the direction of the lower part of the eye surface of eye 1, then observes the lipid layer mapping image of the irradiated eye surface through the slit lamp microscope optical body 8, and calculates the lipid layer thickness of the irradiated part through different image colors.

For those skilled in the art, obviously, the present patent is not limited to the details of the above exemplary embodiments (for example, it is installed on the slit lamp microscope optical body, or on the connecting part/arm of the slit lamp body, or on other additional devices of the slit lamp body), and the patent can be realized in other specific forms without departing from the spirit or basic features of the patent. Therefore, no matter from which point of view, the embodiment should be regarded as exemplary and practicalWithout limitation, the scope of the patent is limited by the appended claims rather than the above description. Therefore, it is intended to include all changes within the meaning and scope of the equivalent elements of the claims in the patent. Any reference numerals in the claims shall not be regarded as limiting the claims involved.

In addition, it should be understood that although the description is described according to the embodiments, not each embodiment contains only one independent technical solution. This description of the description is only for clarity. Those skilled in the art should take the description as a whole, and the technical solutions in each embodiment can also be combined appropriately, form other embodiments that can be understood by those skilled in the art.

We claim:

1. An illumination device (100) for tear film lipid layer on ocular surface attached to an Ophthalmic Slit lamp Microscope, including ophthalmic slit lamp microscope, characterized in that:
a. a left and right rotation mechanism (7) is hinged on an optical body (8) of the opthalmic slit lamp microscope;
b. the illumination device (100) for tear film lipid layer on ocular surface is installed on the left and right rotating mechanism (7);
c. the illumination device (100) for tear film lipid layer on ocular surface comprises a connecting part and an illuminating body, one end of the connecting part is connected with the left and right rotation mechanism (7), the other end of the connecting part is connected with the illuminating body, and light source of the illuminating body is a surface light source; and
d. a front and rear adjusting mechanism (6) is installed on the left and right rotation mechanism (7), and a dry eye device (2) is installed on a sliding part of the front and rear adjusting mechanism (6).

2. The illumination device (100) for tear film lipid layer on ocular surface attached to an Ophthalmic Slit lamp Microscope according to claim 1, wherein an optical direction (9) emitted by a light source of the eye surface tear film lipid layer illumination device is inclined at an inclination angle relative to eye axis direction (10), said inclination angle is ≥ 20°.

3. The illumination device (100) for tear film lipid layer on ocular surface attached to an Ophthalmic Slit lamp Microscope according to claim 2, wherein the illuminating body further comprises a translucent flood board (3), a lipid layer lighting device/LED matrix lamp group (4) and a lipid layer lighting device housing (5), and the lipid layer lighting device/ LED matrix lamp group (4) is installed on an inner surface of the lipid layer lighting device housing (5), the translucent flood plate (3) is installed on an outer surface of the lipid layer lighting device (4).

4. The illumination device (100) for tear film lipid layer on ocular surface attached to an Ophthalmic Slit lamp Microscope according to claim 3, wherein the optical direction (9) emitted by the LED matrix lamp group (4) of the lipid layer lighting device (4) is diffused through the translucent flood board (3), transformed into a soft and uniform area light source, and mapped to the lower half of the cornea on an eye surface.
